(19) United States
(12) Patent Application Publication
(10) Pub. No.: US 2021/0045517 A1
(43) Pub. Date: Feb. 18, 2021

(54) PERSONAL PORTABLE MOTION SENSOR FOR MONITORING THE POSITION AND / OR MOVEMENT OF A SPORTSMAN

(71) Applicant: DASHTAG HOLDING B.V., Eindhoven (NL)

(72) Inventors: Patrick Hamannus SCHUUR, Eindhoven (NL); Cliff Roode, De, Eindhoven (NL)

(21) Appl. No.: 16/328,590

(22) PCT Filed: Aug. 25, 2017
(86) PCT No.: PCT/NL2017/050562

§ 371 Date: Feb. 26, 2019

(30) Foreign Application Priority Data
Aug. 26, 2016 (FR) 2017369

(51) Int. Cl. A45F 5/02; A61B 5/11

(52) U.S. Cl. CPC A45F 5/02; A45F 2005/027; A45F 2200/05; A61B 5/11

(57) ABSTRACT

A personal portable motion sensor 1 has a housing 9, in which there are sensor means, and a frame 13 connected to the housing. Parts of the housing and the frame form clamping parts for releasably clamping the sensor means onto a sportsman's garment. One of the clamping parts 3 is formed by a wall part 7 of the housing and the other clamping part 5 is connected to the frame via a spring 11. The housing is movable relative to the frame between a release position and a clamp position in which the housing is fixed by locking elements with respect to a framework. The housing is hingedly connected to the framework near a first side wall 21 and is provided on one opposite side wall 23 with one of the locking elements 27 which cooperates with the other locking member 29 which forms part of the framework.

### TECHNICAL FILED OF THE INVENTION

The invention relates to a personal portable motion sensor for monitoring the position and/or movement of a sportsman, comprising sensor means, as well as coupling means connected to the sensor means for coupling the sensor means to the sportsman.

### BACKGROUND OF THE INVENTION

Such a motion sensor is well known and is part of a strap that is applied around the body under the sportsman's clothing.

### SUMMARY OF THE INVENTION

It is an object of the present invention to provide a motion sensor of the type defined in the opening paragraph which can be applied more easily. To this end the motion sensor according to the invention is characterized in that the coupling means comprise clamping means for releasably clamping the sensor means on a sportsman's garment. These clamping means comprise two clamping parts which cooperate with each other, of which a first clamping part comprises a wall part of a housing in which housing the sensor means are present, and a second clamping part is connected via a spring to a frame which is also part of the motion sensor. Sensor means are present in the housing. The housing is also connected to the frame and is movable relative to the frame between a release position in which the first clamping part is spaced apart from the second clamping part and a clamping position, in which the two clamping parts are pushed under spring force to each other by the spring. In the clamping position the housing is fixed by fixing means relative to the frame. The motion sensor according to the invention has a small number of components in which various functions are integrated. The housing and coupling means are part of the clamping means and, in addition to containing the motion sensor, respectively, attach to a piece of clothing also the function of releasable clamping of the motion sensor on the piece of clothing.

In an embodiment the clamping parts are provided with teeth to secure the motion sensor more firmly to a piece of clothing. For this purpose in a further embodiment the clamping parts are provided with rubber clamping surfaces.

Yet another embodiment of the motion sensor according to the invention is characterized in that the frame comprises a closed framework and in the clamping position the housing is within the framework and near a first side wall the housing is pivotably connected to the framework. Preferably, the fixing means comprise two mutually cooperating locking elements of which one locking element is present on a second side wall of the housing opposite the first side wall and the other locking element forms part of the framework. So, the fixation means are integrated into the housing and the framework of the motion sensor in an easy way.

A further embodiment of the motion sensor according to the invention is characterized in that lateral parts of the framework extending along both of the other side walls of the housing are provided with hinge openings in which hinge pins are present, which hinge pins are connected to the two other side walls of the housing. At least one of the hinge pins can be pressed and thereby pushed out of the corresponding hinge opening. Because of this construction the housing can be easily removed from the framework.

Preferably, at least a part of the framework is of flexible material. Because of this lateral parts of the framework extending along both of the other side walls of the housing can be pushed towards each other, wherein a bridge part of the framework extending along the second side wall of the housing bends outwardly. As a result, the locking elements can easily be detached from each other to cancel the fixation.

### BRIEF DESCRIPTION OF THE DRAWINGS

The invention will be further elucidated below on the basis of drawings. These drawings show an embodiment of the motion sensor according to the present invention. In the drawings:

FIG. 1 is a perspective view of the motion sensor with the housing in the clamping position;

FIG. 2 is a perspective view of the motion sensor with the housing in release position;

FIG. 3 is a side view of the motion sensor with the housing in release position; and

FIG. 4 is a side view of the motion sensor with the housing in clamping position.

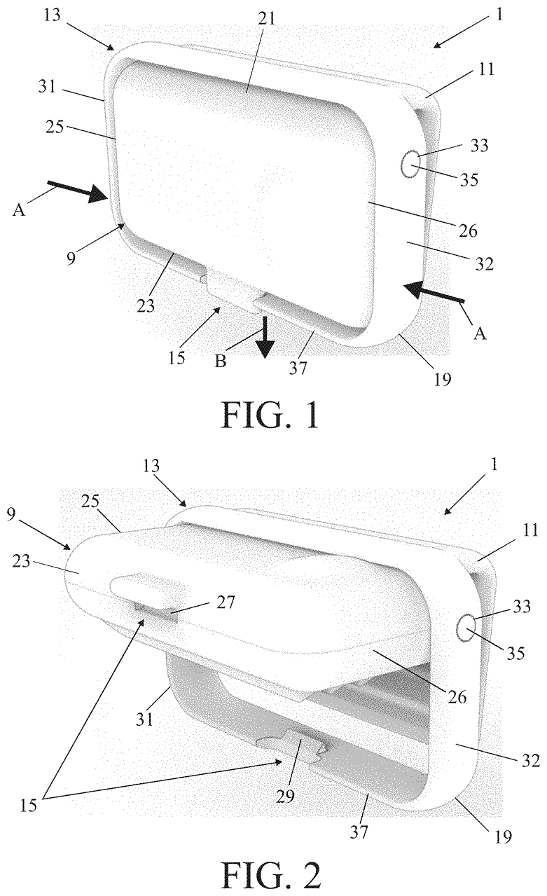

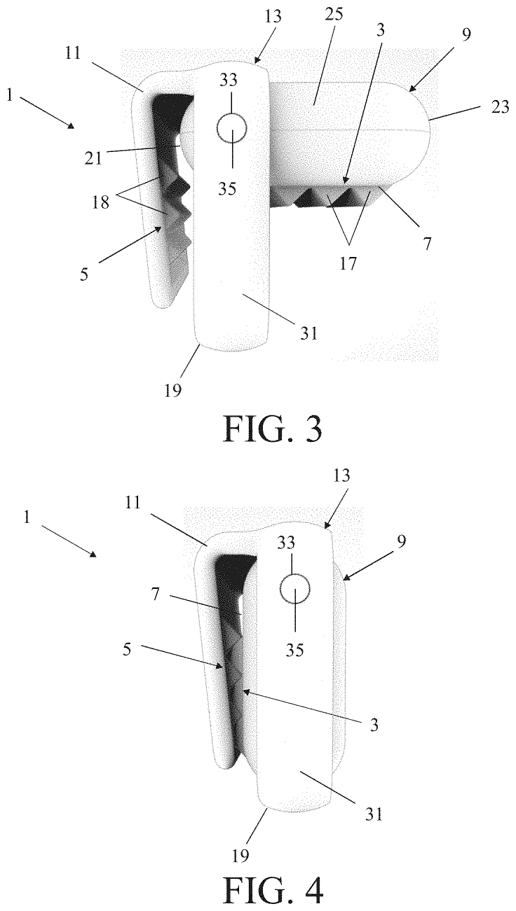

### DETAILED DESCRIPTION OF THE DRAWINGS

A personal portable motion sensor 1 for monitoring the position and/or movement of a sportsman has a housing 9, in which there are sensor means, and a frame 13 connected to the housing which is (partly) around the housing. Parts of the housing and frame form coupling means for coupling the sensor means with the user (sportsman) of the motion sensor.

The coupling means are provided with clamping means for releasably clamping the sensor means on a sportsman's garment. These clamping means have two cooperating clamping parts 3 and 5 provided with rubber teeth 17 and 18. A first of the clamping parts 3 is formed by a wall part 7 of the housing 9 and the other second clamping part 5 is connected via a spring 11 to the frame 13. The housing 9 is movable with respect to the frame 13 between a release position, in which the first clamping part 3 is located at a distance from the second clamping part 5, and a clamping position in which the two clamping parts 3 and 5 are pushed towards each other under spring force by the spring 11. In this clamping position, the housing 9 is fixed by fixing means 15 relative to the frame 13.

The frame 13 has a closed framework 19. In the clamping position the housing 9 is present within this closed framework. Near a first side wall 21 the housing is hingedly connected to the framework and on an opposite second side wall 23 the housing is provided with a locking element 27. This locking element cooperates with a further locking element 29 which forms part of the framework. Both locking elements form the fixing means 15.

The frame 19 further has two lateral parts 31 and 32 extending along the two other side walls 25 and 26 of the housing 9. These lateral parts are provided with hinge openings 33 in which there are hinge pins 35. These hinge pins are connected to both other side walls 25 and 26 of the housing. In order to take the housing 9 out of the frame 19, one of these hinge pins 35 can be pressed so that it gets out of the corresponding hinge opening 33.

The two locking elements 27 and 29 can be made loose from each other by pressing the lateral parts 31 and 32 of the framework 19 in the direction of arrows A to each other. As a result a bridge part 37 of the framework, which extends along the second side wall of the housing and on which the locking element 29 is located, bends outwardly (arrow B). Due to this the locking element gets loose from the other locking element 27.

1. Personal portable motion sensor (1) for monitoring the position and/or movement of a sportsman, comprising sensor means, as well as coupling means connected to the sensor means for coupling the sensor means to the sportsman, characterized in that the coupling means comprise clamping means for releasably clamping the sensor means on a sportsman's garment, which clamping means comprise two clamping parts (3, 5) which cooperate with each other, of which a first clamping part (3) comprises a wall part (7) of a housing (9) in which housing the sensor means are present, and a second clamping part (5) is connected via a spring (11) to a frame (13) which is also part of the motion sensor, to which frame also the housing is connected, wherein the housing (9) being movable relative to the frame (13) between a release position in which the first clamping part (3) is spaced apart from the second clamping part (5) and a clamping position, in which the two clamping parts (3, 5) are pushed under spring force to each other by the spring (11), in which clamping position the housing (9) is fixed by fixing means (15) relative to the frame (13).

2. Motion sensor according to claim 1, characterized in that the clamping parts (3, 5) are provided with teeth (17, 18).

3. Motion sensor according to claim 1, characterized in that the clamping parts (3, 5) are provided with rubber clamping surfaces.

4. Motion sensor according to claim 1, characterized in that the frame (13) comprises a closed framework (19) and in the clamping position the housing (9) is within the framework and near a first side wall (21) the housing is pivotably connected to the framework and at an opposite second side wall (23) the housing is provided with a locking element (27) which cooperates with a further locking element (29) forming part of the framework, which two locking elements are part of the fixing means (15).

5. Motion sensor according to claim 4, characterized in that lateral parts (31, 32) of the framework (19) extending along both of the other side walls (25, 26) of the housing (9) are provided with hinge openings (33) in which hinge pins (35) are present, which hinge pins are connected to the two other side walls (25, 26) of the housing, whereby at least one of the hinge pins (35) can be pressed and thereby pushed out of the corresponding hinge opening (33) to take the housing (9) from the framework (19).

6. Motion sensor according to claim 4, characterized in that lateral parts (31, 32) of the framework (19) extending along both of the other side walls (25, 26) of the housing (9) are of flexible material and can be pushed towards each other, wherein a bridge part (37) of the framework extending along the second side wall (23) of the housing is also of bendable material and bends outwardly so that the locking elements (27, 29) come apart.
